(19) United States
(12) Patent Application Publication
(10) Pub. No.: US 2002/0004072 A1
(43) Pub. Date: Jan. 10, 2002

(54) "COMPOSITION PROVIDING A SUSTAINED RELEASE OF L-HISTIDINE AND METHODS OF MANUFACTURE THEREOF"

(76) Inventor: PETER G. THOMAS, CHARLOTTESVILLE, VA (US)

Correspondence Address: BELL, BOYD & LLOYD LLC, P.O. BOX 1135, CHICAGO, IL, 60690-1135, US

(21) Appl. No.: 09/306,545

(22) Filed: May 6, 1999

(51) Int. Cl. A61K 9/16; A61K 9/50

(52) U.S. Cl. 424/491; 424/502

(57) ABSTRACT

A controlled or sustained release of L-Histidine is provided by coating the L-Histidine with a hydrophobic coating or acid-resistant coating such as a glyceride material derived from rapeseed. Other therapeutic amino acids are coated with hydrophobic or acid-resistant coatings for providing a sustained or controlled release in the gastrointestinal tract to avoid overloading the transport system for the amino acid. In the case of L-Histidine, the overloading of the L-system is avoided and a more constant concentration of L-Histidine can be provided in the gastrointestinal tract. Further, because the L-Histidine provided in a controlled or a sustained release form, a lower overall dose may be administered to the patient.

### FIELD OF THE INVENTION

The present invention relates to compositions containing L-Histidine and which provide a sustained or controlled release of L-Histidine. More specifically, the present invention relates to coatings for L-Histidine which result in a sustained or controlled release of L-Histidine in the gastrointestinal tract.

### BACKGROUND OF THE INVENTION

L-Histidine is an essential amino acid and is not manufactured in the human body. Therefore, L-Histidine must be obtained from dietary sources. In addition to L-Histidine's important role in protein assembly and metabolism, L-Histidine is also an effective antioxidant. Accordingly, L-Histidine may be useful in a variety of radical-based disorders. For example, L-Histidine has been demonstrated to be effective in the treatment of infectious diarrhea, inflammatory bowel diseases such as ulcerative colitis and Crohn's disease. Further, L-Histidine has proven to be useful in the treatment of menstrual cramps, endometriosis as well as ischemia-reprefusion injury and microscopic gastric injuries. The effective dosage range of L-Histidine for a 70 kg adult is from 50 mg to 32 grams per day.

However, one problem associated with the administration of L-Histidine to the gastrointestinal tract is the “rate limiting” effect of the transport systems for L-Histidine and other amino acids. Specifically, active amino acid transport systems are characterized by a maximum rate for the transfer of molar quantity and amino acid classes into a cell. Increasing the concentration of the amino acid available for transport once the maximum transport rate has been reached will not result in an additional increase in transport. As a result, the transport systems are “rate limiting”.

Further, other mechanism that exist to maintain appropriate physiological concentration ranges such as renal clearance, metabolic degration will reduce the excess amino acid present. As a result, the excess amino acid that is not being transported due to the rate limiting effect of the transport system is no longer available for the intended transport or therapeutic process. Amino acid transport systems are discussed by Christensen, “Role of Amino Acid Transport and Counter Transport in Nutrition and Metabolism”, Physiological Reviews, Vol. 70, No. 1 (1990).

A computer-generated pharmacocinetics profile of L-Histidine administered in 2 gram doses orally every two hours is illustrated in FIG. 1. It can be clearly seen from FIG. 1 that the concentration of L-Histidine rises dramatically immediately after the dose but then quickly drops off over the subsequent hour. If it were desired to maintain a 10 mg/dl plasma concentration of L-Histidine, it can be seen from FIG. 1 that it is not possible even with dosing as frequently as every 2 hours.

Accordingly, there is a need for a method for providing a sustained-release or a controlled-release formulation of L-Histidine which will not overload the transport system for this amino acid, the L-system. An advantage of a sustained-release or controlled-release formulation of L-Histidine and other amino acids would be that the transport systems for those amino acids would act more efficiently over a long time period by reducing the rapid peak of plasma concentration illustrated in FIG. 1.

A sustained-release feature would also allow more amino acid to be transported into cells over time without requiring the excessive peak concentrations illustrated in FIG. 1. Such peak concentrations could contribute to toxicity. Further, a sustained-release or controlled-release formulation or system would allow other amino acids to be transported by the same transport system. As a result, by not triggering the rate limiting properties of an amino acid transport system, more than one amino acid can be transported at a time thereby lessening the possibility of adverse effects from amino acid imbalance.

Accordingly, there is a need for a sustained-release and/or a controlled-release composition, method or system for L-Histidine and other amino acids.

### SUMMARY OF THE INVENTION

The present invention satisfies the aforenoted need by providing a composition that provides a sustained release of L-Histidine. The composition comprises L-Histidine coated with a hydrophobic coating or an acid-resistant coating.

In an embodiment, the hydrophobic coating comprises resins, sugars, methylcellulose, magnesium styrate, lactose, and cocoa butter.

In an embodiment, the composition comprises a mixture of uncoated L-Histidine and sustained release or controlled release L-Histidine.

In an embodiment, the L-Histidine is coated with glyceride.

In an embodiment, the L-Histidine is hot-melt coated with glyceride.

In an embodiment, the coating is a glyceride derived from rapeseed.

In an embodiment, the coating is a glyceride sold under the trademark COMPRITOL®888.

In an embodiment, the weight percent of COMPRITOL®888 ranges from about 5% to about 20%.

In an embodiment, the L-Histidine has a particle size ranging from about 125 μm to about 500 μm.

In an embodiment, the composition is provided in a tablet form, gelcap form, a powdered form, a drink form such as a sports drink, an oral rehydration solution, eye drops or ophthalmic solutions, intravenous solutions.

In an embodiment, the present invention provides a method of manufacturing an L-Histidine containing composition that provides a sustained release of L-Histidine. The method comprises the steps of providing L-Histidine particles, fluidizing the L-Histidine particles in a gas flow, and introducing a hydrophobic or acid-resistant coating into the gas flow to coat the L-Histidine particles with the glyceride.

In an embodiment, the coating is a glyceride.

In an embodiment, the gas has a temperature ranging from about 100° C. to about 200° C.

In an embodiment, the glyceride is introduced into the gas flow with a nozzle.

In an embodiment, the gas flow has a pressure ranging from 2 bars to 4 bars.

In an embodiment, the glyceride is a glyceride derived from rapeseed.

In an embodiment, the glyceride is sold under the trademark COMPRITOL®888.

In an embodiment, the weight percent of the COMPRITOL®888 ranges from about 5% to about 20%.

In an embodiment, the L-Histidine particles have a particle size ranging from 125 μm to 500 μm.

In an embodiment, the present invention provides a composition for providing a sustained release of an amino acid for therapeutic purposes. The composition comprises said amino acid coated with a glyceride.

In an embodiment, the therapeutic amino acid is hot-melt coated with the glyceride and, in a further embodiment, the glyceride is sold under the trademark COMPRITOL®888.

It is an advantage of the present invention to provide an improved formulation for the controlled or sustained release of L-Histidine.

It is a further advantage of the present invention to provide an improved formulation for the controlled or sustained release of L-Histidine for the treatment or prevention of cardiac diseases and cardiac surgery, central nervous conditions and neurosurgery, infectious diseases, inflammatory conditions, neurodegenerative disorders, cancers, cancer treatments, digestive, metabolic and gastrointestinal disorders, gynecological conditions, joint, muscle and other pains, inflammation, aging and the sequelae of these conditions.

It is a further advantage of the present invention to provide an improved sustained or controlled release of L-Histidine for use as a drug, a medical food, a dietary supplement and/or a nutritional or nutriceutical product.

It is another advantage of the present invention that a lower overall dose of L-Histidine can be administered using the sustained-release and controlled-release formulations of the present invention.

Additional features and advantages of the present invention are described in, and will be apparent from, the detailed description of the presently preferred embodiments and upon reference to the accompanying figures.

### BRIEF DESCRIPTION OF THE DRAWINGS

FIG. 1 illustrates, graphically, a pharmacokinetics profile of an uncoated oral dose of L-Histidine at 2 hour intervals; and

FIG. 2 illustrates, graphically, the dissolution profiles of L-Histidine preparations coated in accordance with the present invention and uncoated L-Histidine preparations.

### DETAILED DESCRIPTION OF THE PRESENTLY PREFERRED EMBODIMENTS

In a preferred embodiment, L-Histidine powder is formulated into a drinkable suspension, solution or emulsion, an eatable powder and in tablet form by first coating the L-Histidine powder with a glyceride material, more specifically, a glyceride material derived from rapeseed and, still more specifically, a glyceride material sold under the trademark COMPRITOL®888 utilizing a hot melt coating process available from Gattefosse Corporation of Lyon, France. The specific process utilized is known as the “Gattecoat Hot Melt Coating Process”. L-Histidine particles, in a size ranging from 125 μm to 500 μm are provided and fluidized in a gas, such as air, at a temperature ranging from 100 ° C. to 200° C., more preferably, about 140° C. The pressure of the fluidizing air can range from 2 to 4 bars, preferably, 3 bars. The temperature of the COMPRITOL®888 coating is preferably about the same temperature as the fluidizing air. The ratio of the L-Histidine powder to the COMPRITOL®888 can range from 80:20 to 95:5. The COMPRITOL®888 is introduced into the fluidizing air flow by way of a spray nozzle.

Table 1 below summarizes the results of the coating levels achieved for two runs. In Run No. 1, the ratio of L-Histidine to COMPRITOL®888 was 93.3:6.7. The temperature of the fluidizing air was 140° C., the pressure of the fluidizing air was 3 bars. The particle sizes were less than 1 mm in diameter. In Run No. 2, the ratio of L-Histidine to Al₂O₃ to COMPRITOL®888 was 89.6:2.8:7.6. Al₂O₃ was introduced to reduce the electrostatic interaction between the particles. Al₂O₃ is a standard tablet excipient. The temperature of the fluidizing air was 140° C.; the pressure of the fluidizing air was 3 bars. The results for Run Nos. 1 and 2 are provided in Table 1.

TABLE 1
Determination of Coating Level Through Histidine
Quantification

| | | | | Recorded | Estimated |
|---|---|---|---|---|---|
| | Theoretical Value | | | Value | Value |
| | L-His- | Compritol ® | | L-His- | Compritol ® |
| | tidine | 888 | Al₂O₃ | tidine | 888 |
| L-Histidine | 100.0% | 0.0% | | 99.4% | 0.0% |
| (n = 2) | | | | | |
| Run No. 1 | 74.6% | 25.4% | | 85.2% | 14.2% |
| Run No. 2 | 89.6% | 7.6% | 2.8% | 90.1% | 6.3% |

The results of Table 1 are based upon a histidine quantification. The results illustrated in Table 2 are based upon a COMPRITOL®888 quantification and, as shown, are in agreement with those of Table 1.

TABLE 2
Determination of Coating Level Though
Compritol ® 888 Determination

| | | % Compritol ® 888 | |
|---|---|---|---|
| | | estimated by | |
| | % Compritol ® | gravimetric difference | % Compritol ® |
| | 888 | from L-His- | 888 |
| | sprayed | dine determination | measured |
| Run No. 1 | 25.4 | 14.2 | 13.1 |
| Run No. 2 | 7.6 | 6.3 | 6.54 |

The particle size distribution of Run Nos. 1 and 2 are shown in Table 3.

TABLE 3
Particle Size Classification

| | Modal size of L-Histidine | Modal size of |
|---|---|---|
| | powder 80 μm | coated material 125 μm |
| Run No. 1 | 75.8% | 125 μm |
| | 15.5% | 315 μm |
| | 19.7% | 80 μm |
| Run No. 2 | 60.4% | 125 μm |
| | 5.2% | 315 μm |
| | 22.6% | 80 μm |

Density measurements were carried out using standard pharmaceutical methods for the measurement of bulk and tapped density. A comparison of the apparent density of the freshly dispensed or bulk powder to that of powder after settling is shown in Table 4.

TABLE 4
Apparent Bulk and Tapped Densities of Coated and
Uncoated L-Histidine

| | Bulk Density | Tapped Density |
|---|---|---|
| L-Histidine | 0.55 | 0.79 |
| Run No. 1 | 0.53 | 0.66 |
| Run No. 2 | 0.51 | 0.66 |

As shown in Table 4, the reduction in density is a result of the coating of the powder with the less dense COMPRITOL®888. The large reduction in tapped density, however, reflects the fact that the particles are lubricated by the COMPRITOL®888 coating and are easily able to slide over one another, allowing them to be packed more densely into a smaller volume. The coated L-Histidine is more hydrophobic than uncoated L-Histidine.

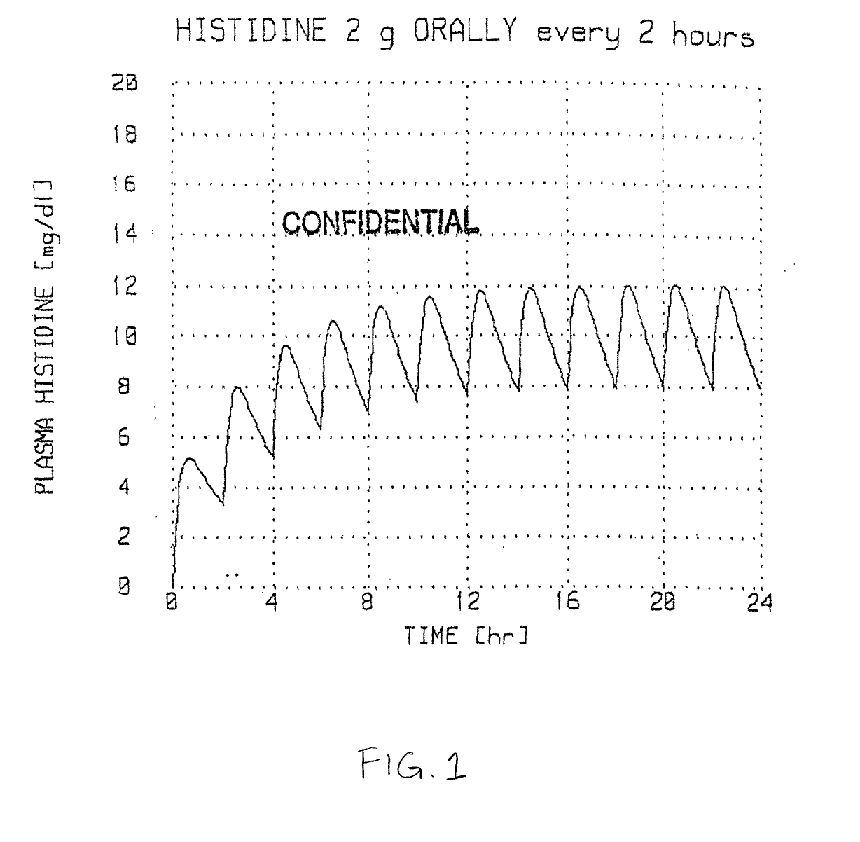

The dissolution of uncoated L-Histidine is illustrated in FIG. 1. Obviously, doses every 2 hours of 2 grams orally do not provide a consistent plasma histidine concentration. The ability of the COMPRITOL®888 coating to slow the dissolution of L-Histidine was carried out using standard pharmacopcal tablet dissolution equipment. Approximately 600 milligrams of coated material were filled into standard gelatin capsules. The capsules were placed in a “sinker”and lowered into a 1 liter chamber of an Erweka DT6 dissolution apparatus. The sample chambers each contained 1 liter of distilled water maintained at 37° C. The chambers were paddle-stirred at 100 rpm. Samples were taken at various time intervals and analyzed for L-Histidine concentration at various time intervals. A further run was carried out in which the capsules were filled with L-Histidine only; in this case, full dissolution of the capsule and contents were observed in under 5 minutes. The run was not analyzed for L-Histidine content since immediate full solubilisation was obvious.

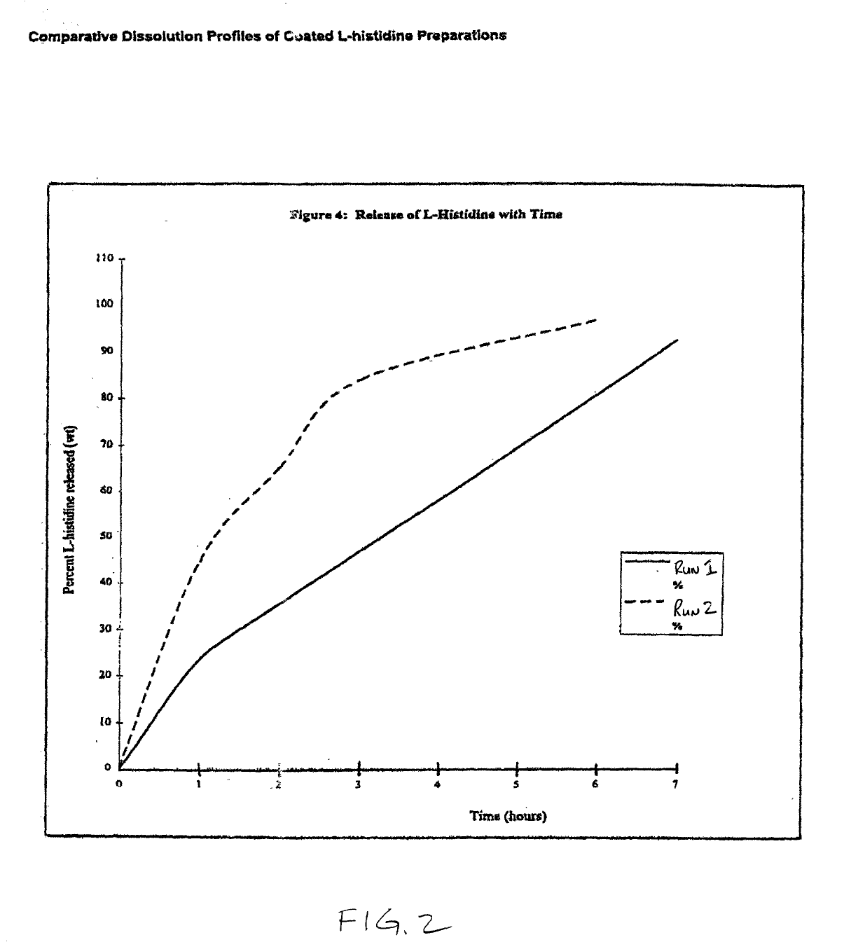

The above parameters, together with the observations of the capsule behavior, are provided in Table 5. The parameters used to calculate the L-Histidine release from the Run Nos. 1 and 2 preparation, are provided in Tables 6 and 7 respectively. The L-Histidine release rate for Nos. 1 and 2 is compared, graphically, in FIG. 2.

TABLE 5
Dissolution Parameters and Description of Experiment

| delta T | | Chamber | Chamber | Chamber | Chamber | Chamber | Chamber | |
|---|---|---|---|---|---|---|---|---|
| hours | Time | 1 | 2 | 3 | 4 | 5 | 6 | Observations |
| Product Weight in | | 0.5393 | 0.5285 | 0.5495 | 0.5244 | 0.5407 | 0.5717 | |
| Capsule | | | | | | | | |
| Calculated Weight | | 0.4627 | 0.4932 | 0.4715 | 0.4914 | 0.4639 | 0.5357 | |
| of L-Histidine | | | | | | | | |
| | 10 h 05 | | | | | | | |
| 0.5 | 10 h 30 | | | | | | | |
| 1 | 11 h 06 | 1 | | 1 | | 1 | | |
| 1 | 11 h 09 | | 1 | | 1 | | 1 | |
| | 11 h 43 | | | | | | | Capsules in Chambers 2, 4 and 6 |
| | | | | | | | | dissolving The remaining |
| | | | | | | | | chambers show no evidence of |
| | | | | | | | | dissolution |
| 2 | 12 h 04 | 2 | | 2 | | 2 | | Capsules in Chamber 1, 3 and 5 |
| | | | | | | | | dissolving |
| 2 | 12 h 08 | | 2 | | 2 | | 2 | |
| 3 | 13 h 05 | | 3 | | 3 | | 3 | Dissolution in Chambers 1, 3 and |
| | | | | | | | | 5 slow |
| | 14 h 05 | | | | | | | |
| 5 | 15 h 05 | | 4 | | 4 | | 4 | |
| 6 | 16 h 05 | 3 | | 3 | | 3 | | |
| 6 | 16 h 08 | | 5 | | 5 | | 5 | Capsules in Chambers 2, 4 and 6 |
| | | | | | | | | completely dissolved Particles |
| | | | | | | | | dispersed |
| 7 | 17 h 05 | 4 | | 4 | | 4 | | In Chambers 1, 3 and 5 the |
| | | | | | | | | powder has remained in the form |
| | | | | | | | | of a capsule Particles have not |
| | | | | | | | | dispersed |
| 7 | 17 h 08 | | 6 | | 6 | | 6 | |
| | 18 h 30 | | | | | | | All material in Chambers 2, 4 and |
| | | | | | | | | 6 dispersed In Chambers 1, 3 |
| | | | | | | | | and 5 the powder has retained the |
| | | | | | | | | shape of the capsule and is not |
| | | | | | | | | dispersed |
| 22, 33 | 8 h 20 | 5 | 7 | 5 | 7 | 5 | 7 | |
| 24 h 00 | 10 h 00 | 6 | 8 | 6 | 8 | 6 | 8 | Some particulate matter still |
| | | | | | | | | present in Chambers 1, 3 and 5 |

NB
An additional run was carried out in which capsules were filled with L-Histidine powder only In all cases, complete dissolution of the capsule and contents were observed in under 5 minutes

TABLE 6
Calculation of L-Histidine Release with Time for Run No. 1
Run No. 1, theoretical weight of L-Histidine present in
the chamber: 0.4627 g

| Time | | | | | | | |
|---|---|---|---|---|---|---|---|
| (h) | μmol % ml | Ci (g/L) | Vi (ml) | Vp (ml) | D | Cf (g/L) | % |
| 0 | 0.0 | 0.000 | 1000 | 1000 | 1.00 | 0.000 | 0.0 |
| 1 | 70.4 | 0.109 | 1000 | 1000 | 1.00 | 0.109 | 23.6 |
| 2 | 107.1 | 0.166 | 1000 | 990 | 1.01 | 0.165 | 35.6 |
| 6 | 245.6 | 0.381 | 1000 | 980 | 1.02 | 0.373 | 80.7 |
| 7 | 284.7 | 0.442 | 1000 | 970 | 1.03 | 0.429 | 92.6 |
| 24 | 321.8 | 0.499 | 1000 | 950 | 1.05 | 0.474 | 102.5 |

TABLE 7
Calculation of L-Histidine Release with Time for Run No. 2
Run No. 2, theoretical weight of L-Histidine present in
the chamber 0.4952 g

| Time | | | | | | | |
|---|---|---|---|---|---|---|---|
| (h) | μmol % ml | Ci (g/L) | Vi (ml) | Vp (ml) | D | Cf (g/L) | % |
| 0 | 0.0 | 0.0 | 1000 | 1000 | 1.00 | 0.0 | 0.0 |
| 1 | 142.2 | 0.2 | 1000 | 1000 | 1.00 | 0.2 | 44.6 |
| 2 | 209.7 | 0.3 | 1000 | 990 | 1.01 | 0.3 | 65.1 |
| 3 | 272.8 | 0.4 | 1000 | 980 | 1.02 | 0.4 | 83.8 |
| 5 | 355.7 | 0.6 | 1000 | 970 | 1.03 | 0.5 | 108.1 |
| 6 | 321.1 | 0.5 | 1000 | 960 | 1.04 | 0.5 | 96.6 |
| 22.5 | 311.7 | 0.5 | 1000 | 940 | 1.06 | 0.5 | 91.8 |

Obviously, the release curves shown in FIG. 2 indicate that coating the L-Histidine has a significant on slowing its dissolution. Furthermore, increasing the level of coating of the L-Histidine increases the degree to which the dissolution of the L-Histidine is retarded.

It is anticipated that an ideal particle size range will fall between 300 and 500 μm.

It is further anticipated that other hydrophobic and/or acid-resistant coatings will be useful as well. Hydrophobic coatings will delay the dissolution of the L-Histidine in the stomach and the gastrointestinal tract. Acid-resistant coatings will similarly delay the dissolution of the L-Histidine in the stomach and gastrointestinal tract.

It should be understood that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims.

What is claimed is:

1. A composition providing a sustained release of L-Histidine, the composition comprising:
L-Histidine coated with a hydrophobic coating.

2. The composition of claim 1 wherein the coating comprises a glyceride.

3. The composition of claim 1 wherein the L-Histidine is hot-melt coated with the glyceride.

4. The composition of claim 3 wherein the glyceride is a glyceride derived from rapeseed.

5. The composition of claim 4 wherein the glyceride is sold under the trademark COMPRITOL®888.

6. The composition of claim 1 wherein the weight percent of the hydrophobic coating ranges from about 5% to about 20%.

7. The composition of claim 1 wherein the L-Histidine has a particle size ranging from about 125 μm to about 500 μm.

8. The composition of claim 6 wherein the L-Histidine has a particle size ranging from about 125 μm to about 500 μm.

9. The composition of claim 1 wherein the composition is provided in a form selected from the group consisting of tablets, gelcaps, powders, oral solutions, nutritional beverages, nutritional bars, intravenous solutions and ophthalmic solutions.

10. A method of manufacturing an L-Histidine-containing composition that provides a sustained release of the L-Histidine, the method comprising the following steps:
providing L-Histidine particles;
fluidizing the L-Histidine particles in a gas flow;
introducing a hydrophobic material into the gas flow to coat the L-Histidine particles with the hydrophobic material.

11. The method of claim 10 wherein the hydrophobic material is a glyceride.

12. The method of claim 10 wherein the gas has a temperature ranging from about 100° C. to about 200° C.

13. The method of claim 11 wherein the glyceride is introduced into the gas flow with a nozzle.

14. The method of claim 10 wherein the gas flow has a pressure ranging from 2 bars to 4 bars.

15. The method of claim 11 wherein the glyceride is a glyceride derived from rapeseed.

16. The method of claim 11 wherein the glyceride is sold under the trademark COMPRITOL®888.

17. The method of claim 16 wherein the weight percent of COMPRITOL®888 ranges from about 5% to about 20%.

18. The method of claim 17 wherein the L-Histidine particles have a particle size ranging from about 125 μm to about 500 μm.

19. The method of claim 10 wherein the L-Histidine has a particle size ranging from about 125 μm to about 500 μm.

20. A composition providing a sustained release of L-Histidine, the composition comprising:
L-Histidine particles coated with a glyceride derived from rapeseed and sol d under the trademark COMPRITOL®888, the weight percent of COMPRITOL®888 ranging from about 5% to about 20%, the particle size of the L-Histidine ranging from about 125 μm to about 500 μm.

21. A composition providing a sustained release of an amino acid, the composition comprising:
coated amino acid particles.

22. The composition of claim 21 wherein the amino acid is L-Histidine and the L-Histidine is coated with a glyceride.

23. The composition of claim 22 wherein the L-Histidine is hot-melt coated with the glyceride.

24. The composition of claim 23 wherein the glyceride is a glyceride derived from rapeseed.

25. The composition of claim 24 wherein the glyceride is sold under the trademark COMPRITOL®888.

26. The composition of claim 25 wherein the weight percent of COMPRITOL®888 ranges from about 5% to about 20%.

27. The composition of claim 26 wherein the amino acid has a particle size ranging from about 125 μm to about 500 μm.

28. A composition providing a sustained release of L-Histidine, the composition comprising:
L-Histidine coated with an acid-resistant coating.

29. A method for providing a therapeutic amount of L-Histidine in a sustained release comprising the step of administering L-Histidine coated with a hydrophobic coating.

30. A method for providing a therapeutic amount of L-Histidine in a sustained release comprising the step of administering L-Histidine coated with an acid-resistant coating.

31. A method for providing a controlled concentration of L-Histidine in blood comprising the step of administering L-Histidine coated with a hydrophobic coating.

32. A method for providing a controlled concentration of L-Histidine in blood comprising the step of administering L-Histidine coated with an acid-resistant coating.
